(19) United States
(12) Patent Application Publication
(10) Pub. No.: US 2025/0214926 A1
(43) Pub. Date: Jul. 3, 2025

(54) METHOD FOR MANUFACTURING COMPOSITE OF HYDROTALCITE COMPOUND AND AMINO ACID

(71) Applicant: SETOLAS Holdings, Inc., Takamatsu-shi, Kagawa (JP)

(72) Inventor: Hiroyuki SUGIYAMA, Sakaide-shi (JP)

(73) Assignee: SETOLAS Holdings, Inc., Takamatsu-shi, Kagawa (JP)

(21) Appl. No.: 18/853,319

(22) PCT Filed: Apr. 3, 2023
(86) PCT No.: PCT/JP2023/013845

§ 371 Date: Oct. 1, 2024

(30) Foreign Application Priority Data
Apr. 13, 2022 (JP) 2022-066619

(51) Int. Cl. C07C 227/18

(52) U.S. Cl. CPC C07C 227/18

(57) ABSTRACT

The present invention provides a method for uniformly and efficiently manufacturing a composite of a hydrotalcite compound and an amino acid with the resulting composite having a high aspect ratio. The present disclosure is a method for manufacturing a composite of a hydrotalcite compound and an amino acid. The composite has an aspect ratio of 85 or higher. The manufacturing method includes a solution adjustment step and a heating step. The solution adjustment step is a step of preparing a slurry solution. The slurry solution is obtained by mixing an aqueous solution of the amino acid and a complex metal oxide. The complex metal oxide is obtained by firing a precursor hydrotalcite compound. The heating step is a step of heating the slurry solution. In the solution preparation step, the slurry concentration is less than 30 g/L. The slurry concentration is the concentration of the slurry solution.

### FIELD

The present invention relates to a method of producing a complex of a hydrotalcite compound and an amino acid.

### BACKGROUND

Hydrotalcite compounds, including hydrotalcite and calcinated products thereof, exhibit ion exchange capacity and are used for a variety of purposes, such as being combined with resins to reduce resin degradation. Such hydrotalcite compounds are used in known methods that utilize amino acids to further increase the aspect ratio (PTL 1 and NPL 1). With the methods disclosed in PTL 1 and NPL 1 it is possible to obtain complexes of hydrotalcite compounds and amino acids.

### CITATION LIST

### Patent Literature

[PTL 1] International Patent Publication No. WO2019/092453

### Non Patent Literature

[NPL 1] Nature Communications (2019) 10:2398, High gas barrier coating using non-toxic nanosheet dispersions for flexible food packaging film.

### SUMMARY

### Technical Problem

With the methods disclosed in PTL 1 and NPL 1, however, gelling can occur during heat treatment of a mixture of a complex metal oxide obtained by calcinating a hydrotalcite compound, and an aqueous solution of glycine as an amino acid, while coloration may occur as well. When gelling of the mixture occurs, the complex of the hydrotalcite compound and amino acid cannot be produced uniformly and efficiently, often causing problems such as loss of quality stability of the complex of the hydrotalcite compound and amino acid.

It is an object of the present invention to provide a method which allows production of a complex of a hydrotalcite compound and an amino acid having a high aspect ratio, in a uniform and highly efficient manner.

### Solution to Problem

As a result of much ardent research with the goal of achieving the object stated above, the present inventors have found that in a method for producing a complex of a hydrotalcite compound and an amino acid, defining a specific range for the concentration of a slurry solution (slurry concentration) obtained by mixing an amino acid aqueous solution with a complex metal oxide obtained by calcinating a hydrotalcite compound precursor, can reduce gelling of the mixture of the complex metal oxide and amino acid aqueous solution during its heat treatment.

The present invention has been completed based on this finding and includes the following aspects.

### (First Disclosure)

The first disclosure is a method for producing a complex of a hydrotalcite compound and an amino acid. The complex has an aspect ratio of 85 or higher. The production method comprises a solution preparation step and a heating step. The solution preparation step is a step in which a slurry solution is prepared. The slurry solution is a mixture of the amino acid aqueous solution and the complex metal oxide. The complex metal oxide is obtained by calcinating a hydrotalcite compound precursor. The heating step is a step in which the slurry solution is heated. The slurry concentration in the solution preparation step is lower than 30 g/L. The slurry concentration is the concentration of the slurry solution.

### (Second Disclosure)

The second disclosure is the first disclosure wherein the amino acid is an amino acid having a solubility of 10 g/100 mL·H₂O or greater.

### (Third Disclosure)

The third disclosure is the first disclosure or second disclosure wherein the amino acid is a glycine compound.

### (Fourth Disclosure)

The fourth disclosure is any one of the first disclosure to third disclosure wherein the hydrotalcite compound is represented by the following formula (1).

$${( {M}^{2 +} )}_{1 - X} {( {M}^{3 +} )}_{X} {( OH )}_{2} {( {A}^{n -} )}_{X / n} \cdot {mH}_{2} O \qquad ( 1 )$$

(In formula (1), M²⁺ represents a divalent metal cation. M³⁺ represents a trivalent metal cation. Aⁿ⁻ represents an n-valent anion. X is a number satisfying the inequality 0.17<X<0.36. In formula (1), n is an integer of 1 to 6, and m is a number satisfying the inequality 0<m<1.80.)

### (Fifth Disclosure)

The fifth disclosure is any one of the first disclosure to fourth disclosure wherein the amino acid concentration is lower than 0.6 mol/L. The amino acid concentration is the amino acid concentration of the slurry solution.

### Advantageous Effects of Invention

The production method of the invention allows production of a complex of a hydrotalcite compound and an amino acid having a high aspect ratio, in a uniform and highly efficient manner.

### BRIEF DESCRIPTION OF DRAWINGS

FIG. 1 is a set of photographs showing slurry solutions after the heating step for Example 1 to Example 3 of the invention and Comparative Example 1 to Comparative Example 3, and photographs of slurry solutions after heating to 100° C. in the heating step, for Comparative Example 4 to Comparative Example 6.

### DESCRIPTION OF EMBODIMENTS

The method for producing a complex of a hydrotalcite compound and an amino acid according to the invention will now be described in detail by preferred embodiments. As used herein, “method for producing a complex of a hydrotalcite compound and an amino acid” will also be referred to simply as “method for producing a complex”. The phrase “complex of a hydrotalcite compound and an amino acid”, for the purpose of the present specification, means that the amino acid(s) chemically modify the hydrotalcite compound, and it may also be referred to hereunder simply as “complex”.

### [Method for Producing Complex]

The method for producing a complex according to one embodiment of the invention is a method for producing a complex of a hydrotalcite compound and an amino acid having an aspect ratio of 85 or higher. The method for producing the complex of the embodiment comprises a solution preparation step, in which a slurry solution comprising a mixture of an amino acid aqueous solution and a complex metal oxide obtained by calcinating a hydrotalcite compound precursor, are mixed; and a heating step in which the slurry solution is heated. In the method for producing the complex of the embodiment, the slurry concentration of the slurry solution in the solution preparation step is lower than 30 g/L.

In the aforementioned solution preparation step of the method for producing the complex of the embodiment, the concentration of the slurry solution (slurry concentration) obtained by mixing an aqueous solution of an amino acid (such as glycine) as the release agent with a complex metal oxide obtained by calcinating a hydrotalcite compound precursor, is within the specific range of lower than 30 g/L. The method for producing the complex of the embodiment can thereby reduce gelling of the mixture during the heating step in which the mixture of the complex metal oxide and the glycine aqueous solution is heated. As a result, the method for producing the complex of the embodiment allows production of a complex of a hydrotalcite compound and an amino acid having a high aspect ratio, in a uniform and highly efficient manner.

In the solution preparation step described above for the method for producing the complex of the embodiment, the complex metal oxide obtained by calcinating the hydrotalcite compound precursor may be hydrated using an amino acid aqueous solution. The amino acid aqueous solution may be an aqueous glycine solution, for example. During hydration, the amino acid molecules in the solution, being anions, become intercalated between the layers of the hydrotalcite compound, causing detachment between the layers of the hydrotalcite compound. Detachment between the layers of the hydrotalcite compound results in formation of particles to a very small thickness. The particles with small thickness are heated in the heating step, forming a complex of a hydrotalcite compound in which growth of the particles is promoted in the widthwise direction. In other words, particles with a high aspect ratio are formed. The complex obtained in this manner, having a high aspect ratio, has multiple complex molecules arranged in parallel in the in-plane direction of the covering layer within the covering layer, after combination with the polymer in the coating solution and formation of the covering layer on the substrate, thus allowing an excellent gas barrier property to be exhibited.

Each of the steps in the method for producing the complex of the embodiment will now be described.

### (Solution Preparation Step)

In the solution preparation step, first the hydrotalcite compound precursor is calcinated to obtain a complex metal oxide. The calcination time for calcination of the hydrotalcite compound precursor is not particularly restricted, and for calcination using a calcination furnace it may be a time of 0.1 to 24 hours, for example. The calcination temperature is also not particularly restricted, and for calcination using a calcination furnace it may be a temperature of 300° C. to 700° C., for example. The calcination time for calcination of the hydrotalcite compound precursor may be a time of 1 minute or longer and 12 hours or less for calcination using microwaves, for example. When the calcination uses microwaves, it may be carried out at a temperature of 300° C. to 700° C., for example.

An amino acid aqueous solution at a prescribed concentration is added to and mixed with the complex metal oxide powder obtained by calcination of the hydrotalcite compound precursor, to obtain a slurry solution. The slurry concentration of the slurry solution is lower than 30 g/L. Since the hydrotalcite compound obtained using such a release agent normally has a small primary particle thickness of several nm, a high slurry concentration often results in gelation during the detaching process, producing a non-homogeneous sample. In the production method of the embodiment, the slurry concentration during the solution preparation step may be set to lower than 30 g/L to reduce interaction between the particles of the hydrotalcite compound, thus helping to inhibit gelation during the detaching process. The production method can therefore maintain a slurry state for the slurry solution, so that a complex can be homogeneously and effectively produced.

The slurry concentration during the solution preparation step is more preferably in the range of 10 g/L to 28 g/L from the viewpoint of productivity. The slurry concentration can be determined by the following formula.

Slurry concentration (g/L)=Complex metal oxide weight (g)/slurry volume (L)

### (Hydrotalcite Compound)

The hydrotalcite compound precursor to be used in the method for producing the complex of the embodiment is not particularly restricted, and for example, it may be a hydrotalcite compound represented by the following formula (1).

$${( {M}^{2 +} )}_{1 - X} {( {M}^{3 +} )}_{X} {( OH )}_{2} {( {A}^{n -} )}_{X / n} \cdot {mH}_{2} O \qquad ( 1 )$$

(In formula (1), M²⁺ represents a divalent metal cation, M³⁺ represents a trivalent metal cation, and Aⁿ⁻ represents an n-valent anion. X is a number satisfying the inequality 0.17<X<0.36. n is an integer of 1 to 6, and m is a number satisfying the inequality 0<m<1.80.)

In formula (1), M²⁺ is preferably Mg²⁺, and M³⁺ is preferably Al³⁺. Because these hydrotalcite compounds are highly safe for the human body and have refractive indexes similar to those of resins such as polypropylene or polyethylene, they have the advantage of helping to maintain transparency when forming covering layers on substrates made of such resins. The molar ratio of Mg/Al₂ is preferably in the range of 4 to 8 from the viewpoint of more reliably obtaining a hydrotalcite structure.

The type of anion for Aⁿ⁻ in formula (1) is not particularly restricted, and it may be carbonate ion (CO₃²⁻) or hydroxide ion (OH⁻), for example. The hydrotalcite compound precursor is preferably carbonate ion from the viewpoint of avoiding generation of corrosive gases such as chlorine gas and nitrogen dioxide gas during calcination of the hydrotalcite compound precursor, i.e. the hydrotalcite compound before calcination.

The aforementioned hydrotalcite compounds are hydrotalcite compound precursors to be used as starting materials in the method for producing the complex of the embodiment, but the same applies for the hydrotalcite compounds included in the complex obtained by the production method. Hydroxide ion is preferred from the viewpoint of obtaining a complex with a high aspect ratio.

### (Amino Acids)

The amino acids to be used in the method for producing the complex of the embodiment are not particularly restricted and may be any types of amino acids such as α-amino acids, β-amino acids or γ-amino acids, for example. More specific examples include aspartic acid, glutamic acid, asparagine, serine, glycine, β-alanine, β-aminobutyric acid, γ-aminobutyric acid and β-leucine. The amino acids used may be of a single type of amino acid, or two or more types of amino acids in combination.

Preferred among the aforementioned amino acid types, as amino acids to be used in the method for producing the complex of the embodiment, are amino acids with a solubility of 10 g/100 mL H₂O or greater, and especially glycine. Such amino acids, and especially glycine, have high permittivity and are therefore advantageous for obtaining a complex of primary particles with small thickness. Glycine which exhibits bacteriostatic action is used as a supplement, coloring agent and aromatic component, and is advantageous in terms of biostability.

The phrase “solubility of 10 g/100 mL H₂O or greater” when used herein means solubility such that the mass of substance (amino acid) dissolving in 100 g of water at 25° C. is 10 g or greater.

These amino acids are amino acids to be used as starting materials in the method for producing the complex of the embodiment, but the same applies for amino acids included in the complex that is obtained by the production method. However, the amino acids in the complex may also include multimers (i.e. peptides) obtained by bonding of multiple different amino acids which may form during the production process. As used herein, the term “amino acid compound” will be used to refer to substances including the aforementioned amino acids and amino acid multimers. For example, a substance including glycine and polyglycine will be referred to as a “glycine compound”.

The amino acid concentration of the slurry solution in the solution preparation step is not particularly restricted but is preferably lower than 0.6 mol/L. If the amino acid concentration of the slurry solution is within this range, it will be easier to obtain a complex not only having an aspect ratio of 85 or higher but also a Y.I. value (representing the yellowness index of the complex) of 0 to 5. When such a complex has been mixed into a coating solution together with a polymer to form a covering layer on a substrate, it becomes possible to form a covering layer that not only has a high gas barrier property but also exhibits high transparency without coloration such as yellowish-brown coloration. The amino acid concentration of the slurry solution is more preferably in the range of 0.2 mol/L to 0.5 mol/L.

In the solution preparation step, the molar ratio between the amino acids in the slurry solution and the trivalent metal cation of the hydrotalcite compound (the molar ratio of amino acid/(M³⁺)₂) is preferably in the range of 2 to 6. For example, when the amino acids are glycine and the trivalent metal cation of the hydrotalcite compound is Al³⁺ the molar ratio of glycine/Al₂ is preferably in the range of 2 to 6. If the molar ratio is within this range, it will be easier to obtain a complex not only having an aspect ratio of 85 or higher but also a Y.I. value (representing the yellowness index of the complex) of 0 to 5. When such a complex has been mixed into a coating solution together with a polymer to form a covering layer on a substrate, it becomes possible to form a covering layer that not only has a high gas barrier property but also exhibits high transparency without coloration such as yellowish-brown coloration.

### (Heating Step)

In the heating step, the slurry solution obtained in the solution preparation step is heated to promote particle growth of the hydrotalcite compound. The amino acid molecules in the solution are incorporated as anions between the hydrotalcite compound layers, causing the hydrotalcite compound layers to detach and forming a complex of the hydrotalcite compound and amino acids as thin particles (i.e., particles with a high aspect ratio).

Heating in the heating step is preferably carried out while stirring the slurry solution. The stirring means is not particularly restricted, but preferably it allows uniform stirring to be accomplished while maintaining the fluidity of the slurry solution.

The heating temperature in the heating step is not particularly restricted but is preferably a temperature in the range of 20° C. to 250° C., with a more preferred upper limit for the heating temperature being 170° C. This will more reliably inhibit degeneration of the amino acids. The heating time is not particularly restricted, but it is preferably a time in the range of 1 minutes to 100 hours. The heating step is preferably carried out in a closed system from the viewpoint of helping to prevent variation in the concentration of the slurry solution.

### (Other Steps)

The steps following the heating step are not particularly restricted so long as the effect of the invention is not hindered. Examples of steps after the heating step include a washing step in which the slurry solution is washed with an alkali solution (such as a sodium hydroxide solution) after the heating step, a solid-liquid separation step in which solid-liquid separation treatment is carried out after the washing step to obtain a solid, a drying step in which the solid obtained by the solid-liquid separation step is dried to obtain a powder of the complex, and a surface treatment step in which the surfaces of the complex particles are treated with any of various surface treatment agents.

A dilution step may also be carried out before the washing step for dilution of the heated slurry with ion-exchanged water, or a coating solution preparation step may be carried out instead of the drying step, for mixing with the polymer solution to prepare a coating solution.

The complex obtained by the production method of the embodiment may be mixed with a polymer solution to prepare a coating solution. The coating solution may be coated and dried on a substrate to form a covering layer that exhibits a high gas barrier property.

The complex obtained by the production method of the embodiment will now be described.

### [Complex]

The complex obtained by the production method of the embodiment is a complex of a hydrotalcite compound and an amino acid, having an aspect ratio of 85 or higher. The amino acid concentration of the slurry solution is preferably adjusted during production, so that the complex has a Y.I. value (representing the yellowness index) of 0 to 5 and an amino acid content of greater than 0 mass % and 10.0 mass % or lower with respect to the total mass of the complex. From the viewpoint of helping to form a covering layer exhibiting both high gas barrier properties and transparency, the amino acid content is preferably 1.0 mass % to 8.0 mass % and even more preferably 1.5 mass % to 6.0 mass %.

If the Y.I. value of the complex is within the specific range of 5 or lower and the amino acid content is within the specific range of 10.0 mass % or lower, it will be possible to form a covering layer with low coloration such as yellowish-brown coloration and high transparency, when the complex is used in the coating solution to form the covering layer. Without being constrained by theory, it is thought that the action mechanism by which a highly transparent covering layer is obtained may be described as follows.

First, the method for producing the complex of the embodiment is advantageous in that using an amino acid in the release agent produces interlayer detachment of the hydrotalcite compound, thereby producing a hydrotalcite compound having a small thickness with primary particles. However, in the heating step in which the hydrotalcite compound is detached, peptide bonding between the amino acids is accelerated under alkaline conditions, forming polyamino acids (for example, peptides such as polyglycine). Peptides such as polyglycine produce yellowing with extended chains, causing yellowness to increase as the molecular chains lengthen. When a complex of a hydrotalcite compound and amino acids that include such long-chain peptides (polypeptides) is combined with a polymer in a coating solution, the transparency and aesthetic quality of the resulting covering layer can potentially be impaired.

By thus specifying ranges for limiting the slurry concentration of the slurry solution to lower than 30 g/L and the amino acid concentration to lower than 0.6 mol/L in the solution preparation step, it is possible to prevent formation of peptide bonds between the amino acids even under alkaline conditions, so that production of polyamino acids (peptides) can be inhibited. As a result, it is possible to reduce yellowing in the obtained complex. By adjusting the slurry concentration of the slurry solution and the amino acid concentration, it is possible to obtain a complex with a high aspect ratio of 85 or higher, while having a Y.I. value (yellowness index) of 0 to 5 and an amino acid content of greater than 0 mass % and 10.0 mass % or lower. Such a complex may be mixed into a coating solution together with a polymer to form a covering layer that when formed, exhibits both a high gas barrier property and high transparency. The complex obtained by the production method of the embodiment can thus be used for a wide variety of purposes that require gas barrier properties, without sacrificing transparency or aesthetic quality. For example, when a coating solution containing such a complex is applied onto a film, it can impart a high gas barrier property to the film without impairing the transparency.

As mentioned above, the complex preferably has a Y.I. value of 0 to 5, as the value representing the yellowness index. The Y.I. value is an index of the “polyamino acid content” which affects coloration such as yellowing. If the Y.I. value is higher than 5, yellowish-brown and similar coloration can occur, potentially impairing the transparency and aesthetic quality when the covering layer is formed using the colored complex as a component of the coating solution. A more preferred range for the Y.I. value is 4 or lower, being even more preferably 3 or lower. If the Y.I. value is within this range it will be possible to form a more highly transparent covering layer.

Since the complex obtained by the production method of the embodiment has a high aspect ratio of 85 or higher, it can exhibit an excellent gas barrier property when combined with a polymer in a coating solution and used to form a covering layer on a substrate. The aspect ratio of the complex is preferably 90 or higher and even more preferably 100 or higher from the viewpoint of a higher gas barrier property. The upper limit for the aspect ratio of the complex is not particularly restricted but may be 500 or lower, for example.

For the present purpose, the aspect ratio of the complex is the ratio between the primary particle width (diameter) and thickness in the complex with a layered structure, and it can be determined by dividing the width of the primary particles in the complex by the thickness.

If the complex obtained by the production method of the embodiment has an aspect ratio of 85 or higher, the primary particle thickness is not particularly restricted but may be a thickness of 20 nm or smaller, for example. After having been combined with the polymer in the coating solution and used to form the covering layer on a substrate, the complex preferably has a complex primary particle thickness of 20 nm or smaller and an aspect ratio of 100 or higher, from the viewpoint of obtaining a superior gas barrier property.

The complex primary particle thickness is more preferably in the range of 0.7 nm to 10 nm. If the primary particle thickness in the complex is within this range, then the hydrotalcite compound will be sufficiently detached and will be able to exhibit a higher gas barrier property after having been combined with the polymer in the coating solution and used to form the covering layer on a substrate.

### [Coating Solution]

The coating solution comprising the complex obtained by the production method of the embodiment and a polymer can be coated and dried on a substrate as described below, to form a covering layer. The concentration of the complex in the coating solution is not particularly restricted, but from the viewpoint of coatability onto substrates it is preferably a concentration of 20 mass % or lower and more preferably 10 mass % or lower.

### (Polymer)

The polymer in the coating solution may be used as a solution dissolved in a solvent such as water or an alcohol. The polymer concentration in the solution is not particularly restricted, but from the viewpoint of coatability onto substrates it is preferably a concentration of 20 mass % or lower and more preferably 10 mass % or lower.

The type of polymer in the coating solution is not particularly restricted, and may be a polymer suited for the purpose of the covering layer or film to be formed. Examples of such polymers include water-soluble polymers, and more specifically polyvinyl alcohol, vinyl alcohol-containing copolymers (such as polyethylene vinyl alcohol), carboxymethyl cellulose, polyacrylic acid and polyacrylamide. These water-soluble polymers may be used alone as single types of polymers, or as combinations of two or more types of polymers. Using a water-soluble polymer can facilitate separation between the substrate and the coating layer while increasing the recycling property, thus helping to contribute to achieving SDGs (Sustainable Development Goals) adopted at United Nations summits.

Polyvinyl alcohol is preferably used among the aforementioned water-soluble polymers from the viewpoint of the gas barrier property, transparency and coatability.

### (Other Components)

The coating solution may also comprise other added components in addition to the complex and polymer. Such added components are not particularly restricted, and examples include antioxidants, reinforcing agents, ultraviolet absorbers, pigments, crosslinking agents and flame retardants. Incidentally, any of these added components may be used as single types alone, or two or more different added components may be used in combination.

### [Film]

The coating solution comprising the complex obtained by the production method of the embodiment and a polymer can be coated and dried on a substrate to form a covering layer. The drying conditions may be appropriately set in a temperature range from ordinary temperature to 160° C. for a period from 1 second to 24 hours, for example. If a film-like substrate (hereunder also referred to as “film substrate”) is used as the substrate, it will be possible to obtain a multilayer structure film having the film substrate covered by the covering layer. The covering layer on the substrate may also be detached to obtain a film with a single layer structure comprising the covering layer. A film with a multilayer structure or single layer structure obtained in this manner can be suitably used as an arbitrary type of packaging film, for example.

### (Substrate)

The substrate to be used to form a film is not particularly restricted, and any substrate may be used depending on the purpose of use of the formed film. Examples of such substrates include the film substrates mentioned above, and more specifically, they include resin-based films including polyolefin films such as polyethylene or polypropylene and polyester films such as polyethylene terephthalate; paper; and fiber sheets such as woven fabrics or nonwoven fabrics and knitted fabrics.

When a film substrate is used as the substrate, the thickness is not particularly restricted but is preferably 0.01 μm to 250 μm, and more preferably in the range of 1 μm to 100 μm, for example. The thickness of the covering layer formed by the coating solution is also not particularly restricted and may be a thickness in the range of 0.01 μm to 100 μm, for example.

The film obtained using a complex obtained by the production method of the invention can be used in a wide range of fields including as a food packaging film, beverage packaging film, beverage bottle, drug packaging film, industrial gas barrier film, gas separation film or paper barrier material.

The invention is not restricted to the embodiments described above or the Examples described below, and can incorporate appropriate combinations, substitutions and modifications within ranges that do not fall outside of the object and gist of the invention.

### EXAMPLES

The invention will now be explained in greater detail using Examples and Comparative Examples, with the understanding that the invention is not limited only to these Examples.

### Example 1

### (Production of Hydrotalcite Compound Precursor)

After placing ion-exchanged water in a 1 L volume reaction tank, 160 mL of a 1.5 mol/L magnesium chloride aqueous solution, 120 mL of a 1 mol/L aluminum chloride aqueous solution and a liquid mixture of 90 mL of an 8 mol/L sodium hydroxide aqueous solution with 60 mL of a 1 mol/L sodium carbonate aqueous solution were simultaneously added dropwise while stirring, to obtain a reaction product. The pH during the reaction was 9.5. After washing the obtained reaction product, ion-exchanged water was added to obtain 700 mL of a re-emulsified slurry. The re-emulsified slurry was subjected to hydrothermal treatment for 13 hours at 170° C. The obtained solid was washed with water and then dried for 16 hours at 105° C. and pulverized. The obtained powder was a hydrotalcite compound represented by the chemical formula Mg_{0.67}Al_{0.33} (OH)₂(CO₃)_{0.17}·0.50H₂O. The hydrotalcite compound was used as a hydrotalcite compound precursor for production of a complex.

### (Calcination of Hydrotalcite Compound Precursor)

The obtained hydrotalcite compound precursor was calcinated in an electric furnace for 12 hours at 450° C. to obtain a calcinated product (complex metal oxide).

### (Solution Preparation Step)

A 17.5 g portion of the obtained calcinated product was loaded into a glass beaker, 175 mL of a 2 mol/L glycine aqueous solution was added (corresponding to 26.3 g of glycine powder), and the mixture was stirred to uniformity. The glycine/Al₂ molar ratio of the obtained slurry solution was 5.26. After then adding ion-exchanged water to a total volume of 700 mL, stirring was further carried out to uniformity. The slurry concentration was 25 g/L. The glycine concentration was 0.5 mol/L.

### (Heating Step)

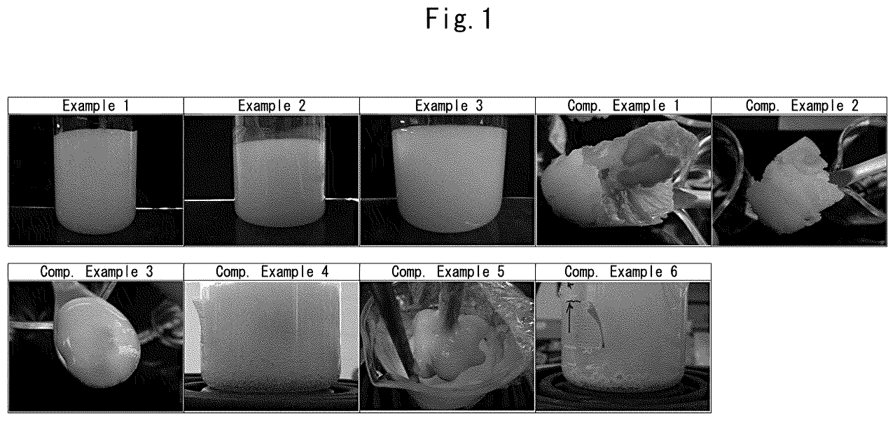

The slurry solution was then stirred at 700 rpm while conducting hydrothermal treatment for 48 hours at 100° C. The obtained sample (slurry solution) was a white slurry with no particular noticeable odor. FIG. 1 shows a photograph of a slurry solution after the heating step.

### (Washing Step to Drying Step)

After adding ion-exchanged water to the obtained slurry solution to a total volume of 1750 mL, a stirrer was used for 16 hours of stirring at 400 rpm, ordinary temperature. While continuing to stir the slurry solution, 88.4 mL of a 3.96 mol/L sodium hydroxide aqueous solution was slowly added dropwise to the slurry solution. The resulting slurry solution was treated for solid-liquid separation to obtain a solid. The obtained solid was dried at 60° C. for 16 hours to obtain a sample (complex) for Example 1. The features of the obtained sample are shown in Table 1.

### Example 2

A sample for Example 2 was obtained in the same manner as Example 1, except that in the solution preparation step, 14.0 g of calcinated product and 140 mL of a 2 mol/L glycine aqueous solution (corresponding to 21.0 g of glycine powder) were used. The slurry concentration of the slurry solution in the solution preparation step was 20 g/L, and the glycine concentration was 0.4 mol/L.

### Example 3

A sample for Example 3 was obtained in the same manner as Example 1, except that in the solution preparation step, 7.0 g of calcinated product and 70 mL of a 2 mol/L glycine aqueous solution (corresponding to 10.5 g of glycine powder) were used. The slurry concentration of the slurry solution in the solution preparation step was 10 g/L, and the glycine concentration was 0.2 mol/L.

### Comparative Example 1

A sample for Comparative Example 1 was obtained in the same manner as Example 1, except that in the solution preparation step, 70.0 g of calcinated product and 700 mL of a 2 mol/L glycine aqueous solution (corresponding to 105.1 g of glycine powder) were used, and stirring was not carried out during the heating step. The slurry concentration of the slurry solution in the solution preparation step was 100 g/L, and the glycine concentration was 2 mol/L.

### Comparative Example 2

A sample for Comparative Example 2 was obtained in the same manner as Example 1, except that in the solution preparation step, 70.0 g of calcinated product and 266 mL of a 2 mol/L glycine aqueous solution (corresponding to 39.9 g of glycine powder) were used, and stirring was not carried out during the heating step. The slurry concentration of the slurry solution in the solution preparation step was 100 g/L, and the glycine concentration was 0.76 mol/L.

### Comparative Example 3

A sample for Comparative Example 3 was obtained in the same manner as Example 1, except that in the solution preparation step, 70.0 g of calcinated product was used, no glycine aqueous solution was used, stirring was not carried out during the heating step, and no washing step was carried out. The slurry concentration of the slurry solution in the solution preparation step was 100 g/L, and the glycine concentration was 0 mol/L.

### Comparative Example 4

A sample for Comparative Example 4 was obtained in the same manner as Example 1, except that in the solution preparation step, 35.0 g of calcinated product and 350 mL of a 2 mol/L glycine aqueous solution (corresponding to 52.5 g of glycine powder) were used. The slurry concentration of the slurry solution in the solution preparation step was 50 g/L, and the glycine concentration was 1 mol/L.

### Comparative Example 5

A sample for Comparative Example 5 was obtained in the same manner as Example 1, except that in the solution preparation step, 28.0 g of calcinated product and 280 mL of a 2 mol/L glycine aqueous solution (corresponding to 42.0 g of glycine powder) were used. The slurry concentration of the slurry solution in the solution preparation step was 40 g/L, and the glycine concentration was 0.8 mol/L.

### Comparative Example 6

A sample for Comparative Example 6 was obtained in the same manner as Example 1, except that in the solution preparation step, 21.0 g of calcinated product and 210 mL of a 2 mol/L glycine aqueous solution (corresponding to 31.5 g of glycine powder) were used. The slurry concentration of the slurry solution in the solution preparation step was 30 g/L, and the glycine concentration was 0.6 mol/L.

### Example 4

### (Preparation of Coating Solution)

The sample of Example 1 and a polyvinyl alcohol (PVA) aqueous solution (Mowiol® 8-88, Mw: 67,000, product of Sigma-Aldrich Japan, KK.) were measured out in solid concentrations, adding ion-exchanged water for preparation to 3 wt % of the complex of the hydrotalcite compound and glycine compound of Example 1 and 2 wt % of PVA, and the mixture was stirred to obtain a coating solution.

### (Fabrication of Film)

First, a polyethylene terephthalate (PET) film (LUMIRROR® by Toray Co., Ltd., film thickness: 50 μm, product number type: #50-U483) cut out to 55 mm length×105 mm width, as a film substrate, was anchored to a glass plate of an automatic coating apparatus (Model PI-1210 by Tester Sangyo Co., Ltd.). A baker applicator (Model YBA by Yoshimitsu Seiki Co.) was then set on the glass plate of the automatic coating apparatus, and the coating solution was coated onto a PET film to a uniform thickness of 50 μm. The covering layer formed by the coated solution was dried for 16 hours at ordinary temperature to obtain a film for Example 4.

### Comparative Example 7

A film for Comparative Example 7 was obtained in the same manner as Example 4, except that the sample of Comparative Example 1 was used instead of the sample of Example 1.

### Comparative Example 8

A film for Comparative Example 8 was obtained in the same manner as Example 4, except that the hydrotalcite compound precursor obtained in the step of “Production of hydrotalcite compound precursor” in Example 1 was used instead of the sample of Example 1 (complex of a hydrotalcite compound and glycine compound). In other words, the film of Comparative Example 8 used an undetached hydrotalcite compound (undetached HT) instead of a complex of a hydrotalcite compound and glycine compound.

### Comparative Example 9

A film for Comparative Example 9 was obtained in the same manner as Example 4, except that no coating solution was applied. In other words, the film of Comparative Example 9 was a simple PET film (film substrate) without formation of a covering layer of a coating solution.

### Comparative Example 10

A film for Comparative Example 10 was obtained in the same manner as Example 4, except that a coating solution comprising PVA alone was used.

### Example 5

### (Fabrication of Film)

A film for Example 5 was obtained in the same manner as Example 4, except that a polypropylene (PP) film (PYLEN® Film-OT by Toyobo, Ltd., film thickness: 50 μm, grade: P2261) was used as the film substrate, and the coating solution was applied to a uniform thickness of 25 μm.

### Example 6

A film for Example 6 was obtained in the same manner as Example 5, except that the coating solution was applied to a uniform thickness of 12.5 μm.

### Comparative Example 11

A film for Comparative Example 11 was obtained in the same manner as Example 5, except that no coating solution was applied. In other words, the film of Comparative Example 11 was a simple PP film (film substrate) without formation of a covering layer of a coating solution.

### Comparative Example 12

A film for Comparative Example 12 was obtained in the same manner as Example 5, except that a coating solution comprising PVA alone was used.

### [Measurements]

The following physical properties of the samples from Example 1 to Example 3 and from Comparative Example 1 to Comparative Example 6 were measured. The measurement results are shown in Table 1 below. Photographs were taken of slurry solutions after the heating step, for Example 1 to Example 3 and Comparative Example 1 to Comparative Example 3, and slurry solutions after heating to 100° C. in the heating step, for Comparative Example 4 to Comparative Example 6. The photographs are shown in FIG. 1.

(Viscosity Measurement after Heating Step)

After adjusting the slurry solution to a temperature of 25° C., a Brookfield viscometer (Model DV2T by Brookfield) was used to measure the viscosity at the same temperature.

### (Measurement of Primary Particle Thickness and Aspect Ratio of Complex)

A scanning probe microscope (Model SPM-9700HT by Shimadzu Corp.) was used to measure the primary particle width and thickness. The aspect ratio was calculated by the following formula.

Primary particle aspect ratio=(Primary particle width)/(primary particle thickness)

The aspect ratio was determined by calculating the aspect ratio for each of 20 arbitrary primary particles, and taking the average as the aspect ratio of the sample.

### (Measurement of Glycine Compound Content)

After mixing 0.5 g of a powder sample dried at 60° C. for 16 hours, 5 g of potassium sulfate and 2 g of copper (II) sulfate pentahydrate and placing the mixture in a sample tube, 15 ml of concentrated sulfuric acid was added. A Kjeldahl Analysis System (KjelFlex K-360/K-425 SpeedDigester by Buchi Co.) was then used for 90 minutes of treatment at 470° C. Next, 10 mL of ion-exchanged water, a 30% sodium hydroxide aqueous solution and 40 mL of a 2% boric acid aqueous solution were used for steam distillation for 240 seconds. Three drops of a mixed indicator of methyl red and methylene blue were added to the obtained distillate, and a 0.1 mol/L hydrochloric acid aqueous solution was used for titration until the distillate turned purple. A blank without the powder sample was also used for titration in the same manner, and the glycine compound content was calculated by the following formulas (I) and (II).

$$Nitrogen N content [ wt \% ] = ( 0.1 mol / L hydrochloric acid solution factor ) \times ( drip volume [ ml ] - blank [ ml ] ) \times ( 1.401 [ mg / ml ] ) \times 100 / powder sample mass [ mg ] \qquad ( I )$$

$$Glycine compund content [ wt \% ] = Nitrogen content [ wt \% ] \times ( 75.07 [ g / mol ] / 14 \text{.01} [ g / mol ] ) \qquad ( II )$$

### (Measurement of Yellowness Index (Y.I. Value))

The powder sample that had been dried at 60° C. for 16 hours was pulverized and screened through a screen net with a 150 μm mesh, after which 0.2 g was placed in a glass reagent bottle. Next, a 10φ viewport and sample stage were used for measurement with a color measurement color meter (Model ZE6000 Color meter by Nippon Denshoku Industries Co., Ltd.) which had been previously calibrated using a standard white board. Measurement was conducted a total of 3 times, shaking the reagent bottle 10 times before each measurement. The average value was calculated for three measurements to obtain the Yellow Index value (Y.I. value).

### [Film Evaluation]

The following physical properties of the films of Example 4 to Example 6 and Comparative Examples 7 to 12 were measured. The measurement results are shown in Table 2 and Table 3 below.

### (Measurement of Total Light Transmittance and Haze (Cloudiness))

The film was cut at 20 mm from the coating end point, and a cloudiness meter (Model NDH4000 by Nippon Denshoku Industries Co., Ltd.) was used for measurement with the coated side as the light-incident side, to obtain the total light transmittance and haze (cloudiness).

### (Measurement of Oxygen Gas Permeability)

The film was punched out to a circular shape with a diameter of about 55 mm, and a gas permeability tester (Model GTR-11AET by GTR Tec Corp.) was used to measure the gas permeability. The measuring method was carried out under the following conditions, according to JIS K 7126-1, Part 1 (“Differential pressure method”).
Measuring temperature: 23.0° C.
Relative humidity: zero
Permeation area: 15.2 cm²
Measuring gas: oxygen gas

The oxygen gas permeating through the film was analyzed using a capillary gas chromatography system (Model GC-2014 by Shimadzu Corp.), to obtain the oxygen gas permeability. For oxygen gas permeability measurement, the analysis time can be appropriately adjusted according to the gas permeability of the film.

TABLE 1

| | | | | | Comp. | Comp. |
|---|---|---|---|---|---|---|
| | | Example 1 | Example 2 | Example 3 | Ex. 1 | Ex. 2 |
| Complex | Glycine concentration | 0.50 | 0.40 | 0.20 | 2.00 | 0.76 |
| production | (mol/L) | | | | | |
| conditions | Glycine/Al₂ molar | 5.26 | 5.26 | 5.26 | 5.26 | 2.00 |
| | ratio | | | | | |
| | Slurry concentration | 25 | 20 | 10 | 100 | 100 |
| | (g/L) | | | | | |
| | Uniformly stirrable or | Yes | Yes | Yes | No | No |
| | not | | | | | |
| State after | Outer appearance | White | White | White | Surface layer | White |
| heating step | | | | | yellowish-brown, | |
| | | | | | rest white | |
| | Viscosity state | Slurry | Slurry | Slurry | Gel | Gel |
| | Viscosity (cP) | 1820 | 504 | 14.6 | Unmeasurable | Unmeasurable |
| | Odor | None | None | None | Burnt odor | None |
| Complex | Primary particle | 2 | 3 | 3 | 3 | 11 |
| characteristics | thickness (nm) | | | | | |
| | Aspect ratio | 171 | 112 | 114 | 223 | 25 |
| | Glycine compound | 4.66 | 5.14 | 4.07 | 5.20 | 4.45 |
| | content (wt %) | | | | | |
| | Y.I. value | 1.7 | 2.3 | 1.0 | 5.6 | 3.3 |

| | | Comp. | Comp. | Comp. | Comp. |
|---|---|---|---|---|---|
| | | Ex. 3 | Ex. 4 | Ex. 5 | Ex. 6 |
| Complex | Glycine concentration | 0.00 | 1.00 | 0.80 | 0.60 |
| production | (mol/L) | | | | |
| conditions | Glycine/Al₂ molar | 0.00 | 5.2 | 5.26 | 5.26 |
| | ratio | | | | |
| | Slurry concentration | 100 | 50 | 40 | 30 |
| | (g/L) | | | | |
| | Uniformly stirrable or | Yes | No | No | No |
| | not | | | | |
| State after | Outer appearance | White | Bottom section | Bottom section | Bottom section |
| heating step | | | yellowish-brown, | yellowish-brown, | yellowish-brown, |
| | | | rest white | rest white | rest white |
| | Viscosity state | Slurry | Gel | Gel | Gel |
| | Viscosity (cP) | 1.2 | Unmeasurable | Unmeasurable | Unmeasurable |
| | Odor | None | — | — | — |
| Complex | Primary particle | 37 | — | — | — |
| characteristics | thickness (nm) | | | | |
| | Aspect ratio | 6 | — | — | — |
| | Glycine compound | 0.00 | — | — | — |
| | content (wt %) | | | | |
| | Y.I. value | 0.9 | — | — | — |

TABLE 2

| | | | Comp. | Comp. | Comp. | Comp. |
|---|---|---|---|---|---|---|
| | | Example 4 | Example 7 | Example 8 | Example 9 | Example 10 |
| Film fabrication | Added complex | Example 1 | Comp. | Undetached | None | None |
| conditions | | | Example 1 | HT | | |
| | Coating thickness (μm) | 50.0 | 50.0 | 50.0 | None | 50.0 |
| Film | Total light transmittance | 91.7 | 91.6 | 70.6 | 92.1 | 91.6 |
| characteristics | (%) | | | | | |
| | Haze (cloudiness X %) | 3.5 | 5.1 | 98.8 | 1.1 | 0.9 |

TABLE 3

| | | | | Comp. | Comp. |
|---|---|---|---|---|---|
| | | Exam- | Exam- | Exam- | Exam- |
| | | ple 5 | ple 6 | ple 11 | ple 12 |
| Film | Added | Exam- | Exam- | None | None |
| fabrication | complex | ple 1 | ple 1 | | |
| conditions | Coating | 25.0 | 12.5 | None | 25.0 |
| | thickness (μm) | | | | |
| Film | O₂ permeability | 0.5 | 0.3 | 802 | 39.6 |
| characteristics | [cm³/(m² · | | | | |
| | day · atm)] | | | | |

As shown in Table 1 to Table 3 and FIG. 1, with the Examples of the invention it was possible to obtain complexes of hydrotalcite compounds and glycine (amino acid) compounds which could be uniformly stirred during the heating step, and which exhibited no gelling and high aspect ratios. it was thus demonstrated that the production methods of the Examples can uniformly and efficiently produce complexes with high aspect ratios. It was further demonstrated that the complexes obtained by the production method of the invention can form films (covering layers) having high gas barrier properties and transparency. With the Comparative Examples, on the other hand, it was not possible to accomplish uniform stirring during the heating step, or gelling occurred, or the aspect ratio of the obtained complexes was lowered, such that it was not possible to uniformly and efficiently produce complexes with high aspect ratios as according to the invention.

### INDUSTRIAL APPLICABILITY

The method for producing a complex of the invention can be suitably used for production of products in a wide range of fields including the fields of food packaging films, beverage packaging films, beverage bottles, drug packaging films, industrial gas barrier films, gas separation films, paper barrier materials, coating materials, scratch resistant materials, cosmetic products and additives.

1. A method of producing a complex of a hydrotalcite compound and an amino acid, having an aspect ratio of 85 or higher, the method comprising:
a solution preparation step in which an amino acid aqueous solution is mixed with a complex metal oxide obtained by calcinating a hydrotalcite compound precursor, to prepare a slurry solution, and
a heating step in which the slurry solution is heated,
wherein a slurry concentration of the slurry solution during the solution preparation step is lower than 30 g/L.

2. The method according to claim 1, wherein the amino acid is an amino acid having a solubility of 10 g/100 mL·H₂O or greater.

3. The method according to claim 1, wherein the amino acid is glycine.

4. The method according to claim 1, wherein the hydrotalcite compound is represented by formula (1),
$${( {M}^{2 +} )}_{1 - X} {( {M}^{3 +} )}_{X} {( OH )}_{2} {( {A}^{n -} )}_{X / n} \cdot {mH}_{2} O \qquad ( 1 )$$
(In formula (1), M²⁺ represents a divalent metal cation, M³⁺ represents a trivalent metal cation, Aⁿ⁻ represents an n-valent anion, X represents a number satisfying 0.17<X<0.36, n is an integer of 1 to 6, and m represents a number satisfying 0<m<1.80).

5. The method according to claim 1, wherein an amino acid concentration of the slurry solution in the solution preparation step is lower than 0.6 mol/L.
